(19) United States
(12) Patent Application Publication
(10) Pub. No.: US 2016/0177257 A1
(43) Pub. Date: Jun. 23, 2016

(54) OPTIMISED METHOD FOR BREAKING CHLORELLA CELL WALLS BY MEANS OF VERY HIGH PRESSURE HOMOGENISATION

(71) Applicant: ROQUETTE FRERES, Lestrem (FR)

(72) Inventor: Samuel PATINIER, Quesnoy-sur-deule (FR)

(21) Appl. No.: 14/905,686

(22) PCT Filed: Jul. 17, 2014
(86) PCT No.: PCT/FR2014/051839

§ 371 Date: Jan. 15, 2016

(30) Foreign Application Priority Data
Jul. 19, 2013 (FR) 1357130

(51) Int. Cl. C12N 1/12; C12N 1/06

(52) U.S. Cl. CPC C12N 1/12; C12N 1/066; A23L 1/3255; A23L 1/025; A23V 2002/00

(57) ABSTRACT

Disclosed is an optimized method for breaking the cell walls of microalgae of the Chlorella genus, more particularly Chlorella vulgaris, Chlorella sorokiniana or Chlorella protothecoides on an industrial scale, implementing very high pressure homogenization technology for processing microalgae biomass.

The present invention relates to an optimized process for breaking the cell walls of microalgae of the Chlorella genus, more particularly Chlorella vulgaris, Chlorella sorokiniana or Chlorella protothecoides on an industrial scale.

### PRESENTATION OF THE PRIOR ART

It is well known to those skilled in the art that chlorellae are a potential source of food, since they are rich in proteins and other essential nutrients.

They contain in particular 45% of proteins, 20% of fats, 20% of carbohydrates, 5% of fibers and 10% of minerals and vitamins.

In order to efficiently use Chlorella in food, use is often made of “cell breaking” so as to facilitate its digestibility and its absorption rate.

This “cell breaking” of the microalgae is well described in the patent and non-patent literature through the use of varied technologies:
physical technologies (ultrasound, microbeads, heat shocks, high pressure, etc.)
chemical technologies (acids, alkalis, hydrophilic organic solvents, etc.)
enzymatic technologies (cellulase, lipase, etc.).

However, the various mechanical, chemical or enzymatic alternatives generally cannot be very successfully extrapolated to an industrial scale and are essentially described on a laboratory scale.

Furthermore, if the cell wall has a particularly high mechanical strength (in particular for the Chlorella genus) and if the cell density in the medium is high (>100 g/l), the choice of technology becomes very limited.

When the industrial problems of extrapolation (high capacity, reliability, operating costs, investment costs, etc.) are also added to this, the realistic mechanical alternatives are essentially restricted to milling using microbeads and to high-pressure technology.

The conventional technologies of cell breaking by high-pressure homogenization, the field addressed by the present invention, are for example described in patent applications or patents CN 102433015 (for the extraction of cyanine from single-cell blue algae), CN 101817738 (for the extraction of DHA from DHA-producing microorganisms), U.S. Pat. No. 5,885,564 (for obtaining oxygen-bearing compositions containing phospholipids and fluorocarbon) or else CN 101352249 (for the extraction of lipid and protein constituents from microalgal biomass).

High-pressure homogenization (termed “HPH” in the remainder of this disclosure), also known as dynamic high-pressure homogenization, has been proposed through the development of a new generation of homogenizers, capable of reaching pressures 10-15 times higher than conventional machines.

Given the nature of the cell material to be “broken”, HPH results in the production of emulsions, composed essentially of a mixture of cell debris, of intracellular aqueous liquid and of oil.

At the beginning of the 1980s, a new technology was proposed for the production of fine emulsions by virtue of the availability of machines capable of generating and managing very high pressures in liquids, pressures greater than 100 MPa and up to 300-500 MPa, and also by virtue of the new design of the homogenization chamber.

Various manufacturers of high-pressure homogenizers thus propose either prototypes or industrial-scale equipment, such as Microfluidics, Stansted Fluid Power, AVP, Avestin or Niro Soavi.

Conceptually, the basic homogenizer is made up of a high-pressure generator, such as a positive displacement pump coupled to a pressure amplifier which forces the fluid through a set of specially designed homogenization valves.

The rapid pressurization of the processing fluid (up to 350 MPa) causes an increase in temperature of about 3° C./100 MPa, while the instantaneous drop in pressure which occurs in the homogenization valve induces a greater increase in heat (15 to 20° C./100 MPa).

A secondary valve, where a much smaller drop in pressure occurs, can be placed alongside the main valve in order to disrupt the agglomerates possibly formed in the first step.

Given that the final temperature may be high, depending on the input temperature and on the operating pressure level, rapid cooling of the processing fluid represents good practice for preserving the thermolabile components of the processed product.

Notably, in these dynamic high-pressure operations, the fluid is exposed to high pressures for very short periods of time (1-10 s).

In this period of time, the fluid must flow from the amplifier to the disruption valve. The breaking operation is mainly regulated by passing the process fluid under high pressure through a discharge valve with an adjustable restricted orifice, rather than by exposure to high pressure.

Whatever the type of homogenization valve, the liquid processed under high pressure passes through a converging section called “the homogenization space”, then dilates.

The pressure is controlled by an actuator, thereby making it possible to adjust the force exerted on the valve.

This HPH technology must therefore be finely controlled in order to be efficient, and must be adjusted according to the intended objectives.

Thus, alongside the use of HPH to break cell walls, for example of microalgae in order to make them more digestible, or to release the content of interest thereof, another application concerns microbial decontamination.

In this field, the company Stansted Fluid Power Ltd has introduced a significant improvement to the design of the homogenizer valve, which makes it possible, by means of increasing the pressure level of the fluid up to 350 MPa, to sterilize products in situ by modifying the texture of emulsions or of biopolymers.

It results from all the aforementioned that HPH technology appears to be promising when effective industrial extrapolation of this cell-breaking technology is intended.

However, when it is necessary to apply it to the breaking of cells of microalgae in general, and of chlorellae in particular, numerous problems arise.

A first problem relating to the processing of Chlorella biomass by HPH is linked to the nature of the emulsion produced.

The stability of this emulsion will depend, inter alia, on the molecules which lie at the interface and also on the emulsification work provided by the homogenization technology.

The stability of said emulsion is generally essential for it to be used in multiple food applications.

A second problem relates to the drying of said emulsion.

Indeed, when this emulsion is dried (for example, by spray drying), the fineness of the emulsion will condition the properties of flow of the powder, of encapsulation of lipids, and of stability with respect to oxidation.

The final product will be all the easier to use according to the intended application.

Depending on the matrix, the energy required for the emulsification may be very high in order to manage to generate a sufficiently fine and stable emulsion.

A third problem is the microbial quality to be observed during the processing for refining/purification of a wet biomass such as a Chlorella fermentation must.

In the case where the refining/purification protocol is carried out in a non-sterile environment, particular precautions are to be taken in order to limit the development of a microbial contamination (for example, refrigerated process, limitation of the intermediate storage phase duration, etc.).

However, these prevention means may be insufficient and a pasteurization/sterilization step may prove to be necessary among the final steps generating the final product.

Faced with these three problems, those skilled in the art will be obliged to have recourse to a laborious process with successive milling, pasteurization/sterilization and homogenization steps with the appropriate technologies.

### SUBJECT OF THE INVENTION

To overcome these constraints, the applicant company chose to carry out its studies on the control of very high (or ultrahigh) pressure technology in order to successfully perform these three actions simultaneously and thus significantly simplify the sequence of operations.

More particularly, the applicant company decided to take advantage of two of the different fields of application of HPH technology, conventionally considered separately by those skilled in the art.

The first field relates to the physical changes which can be brought about by HPH technology, such as the reduction in size and the narrowing of the size distribution of particles, of droplets or of micelles of suspensions or of emulsions, generally described and used for the preparation or the stabilization of emulsions or of preparations of nano particle and nano suspension type, or for the purpose of obtaining changes in viscosity and in texture.

The second field is centered around the effect of cell “disruption” induced by HPH, which is generally applied to the recovery of intracellular material in the biotechnology and pharmaceutical industry, where generally HPH is used to reduce the microbial load in food and pharmaceutical products.

Conducting the HPH in this way thus makes it possible to efficiently use it alone for breaking the microalgal cells, while simultaneously taking advantage of all the functionalities offered by this technology, in order to manage the problems pertaining to:
the production of an emulsion,
the drying of said emulsion
the presence of potential microbial contaminants during the steps of refining/purifying the lysed biomass.

It also makes it possible to dispense with the need to couple the HPH with other technologies, as described in the prior art (such as that using specific enzymes for degrading the cell walls of microalgae), in order to efficiently break the microalgal cells.

Thus, the present invention relates to a process for breaking the cell wall, on an industrial scale, of cells of microalgae of the Chlorella genus, the cell-wall breaking being carried out by high-pressure homogenization, characterized in that the high-pressure homogenization is carried out:
in at least one pass at a pressure between 300 and 400 MPa, or
in at least two successive passes at a pressure between 150 and 300 MPa, and
at a feed temperature between 4 and 40° C.,

in such a way as to guarantee a degree of cell-wall breaking of more than 80%, a particle size of the emulsion produced of less than 1 μm and a decrease in microbial load, in particular by a factor of 1000 or 10 000.

In particular, the process guarantees a microbial load of less than 10 total microorganisms per g of emulsion.

Preferably, the high-pressure homogenization is carried out on a biomass of microalgal cells comprising between 15% and 50% by weight of solids, in particular a biomass of 15% to 50% by dry weight of microalgal cells.

Preferably, the microalgae of the Chlorella genus are chosen from the group consisting of Chlorella vulgaris, Chlorella sorokiniana and Chlorella protothecoides, and are more particularly Chlorella protothecoides. Preferably, the microalgal biomass comprises at least 10% by dry weight of lipids, preferably at least 20%, 30%, 40%, 50% or 60% by dry weight of lipids.

In one particular embodiment, the feed temperature is between 20 and 40° C.

In one preferred embodiment, the high-pressure homogenization is carried out at a pressure between 300 and 400 MPa in a single pass.

In another preferred embodiment, the high-pressure homogenization is carried out at a pressure between 150 and 300 MPa, preferably between 150 and 250 MPa, in two, three, four or five successive passes. In particular, the high-pressure homogenization is carried out at a pressure between 150 and 250 MPa in two successive passes. The pressures of two successive passes may be identical or different.

The present invention also relates to a process for preparing a flour of microalgae, preferably of the Chlorella genus, in particular Chlorella protothecoides, which comprises production of a microalgal biomass, cell-wall breaking by means of a process according to the present invention, and drying of the biomass, in particular by spray drying.

It also relates to the microalgal flour obtained by means of the process. Preferably, the process comprises at least two successive passes at a pressure between 150 and 300 MPa.

Finally, the present invention relates to the use of the process for breaking the cell wall of microalgae according to the present invention, for preparing a microalgal flour as described above.

### DETAILED DESCRIPTION OF THE INVENTION

The applicant company has found that the managed exploitation of the very high (or ultrahigh) pressure homogenization technology for breaking the cell wall of microalgae of the Chlorella genus makes it possible to achieve the desired milling quality.

This managed exploitation is understood to mean the study and optimization of each of the parameters for carrying out the HPH, adapted to the model microalga, in this case Chlorella protothecoides.

The preferred microalgae of the invention can grow in heterotrophic conditions (on sugars as carbon source and in the absence of light). The applicant company recommends choosing lipid-rich microalgae of the Chlorella genus. The microalgae used may be chosen, non-exhaustively, from Chlorella protothecoides, Chlorella kessleri, Chlorella minutissima, Chlorella sp., Chlorella sorokiniama, Chlorella luteoviridis, Chlorella vulgaris, Chlorella reisiglii, Chlorella ellipsoidea, Chlorella saccarophila, Parachlorella kessleri, Parachlorella beijerinkii, Prototheca stagnora and Prototheca moriformis. Preferably, the microalgae used according to the invention belong to the Chlorella protothecoides species.

The microalgae are cultured in liquid medium in order to produce the biomass as such. According to the invention, the microalgae are cultured in a medium containing a carbon source and a nitrogen source in the absence of light (heterotrophic conditions). The solid and liquid growth media are generally available in the literature, and the recommendations for preparing the particular media which are suitable for a large variety of microorganism strains can be found, for example, online at www.utex.org/, a website maintained by the University of Texas at Austin for its culture collection of algea (UTEX). The production of biomass is carried out in fermenters (or bioreactors).

The specific examples of bioreactors, the culture conditions, and the heterotrophic growth and methods of propagation can be combined in any appropriate manner in order to improve the efficiency of the microalgal growth and of the lipids.

Preferably, the biomass to which the very-high-pressure homogenization process is applied has a solids content of between 15% and 50% by dry weight. In particular, the solids content may be between 25% and 45%, preferably between 35% and 45%. Preferably, the biomass is washed and concentrated prior to the application of the very-high-pressure homogenization process. In one preferred embodiment, when a solids content is mentioned, it means a content by dry weight of microalgal cells.

In addition, the lipid content of the microalgal biomass is preferably a minimum of at least 10%, 20%, 30%, 40%, 50% or 60% by dry weight, for example between 20% and 80% or between 30% and 70%.

Evaluation of the Degree of Cell Breaking According to the Pressure Applied, to the Number of Passes and to the Temperature of the Feed Fluid

As will be exemplified hereinafter, it is verified that the pressure applied to the fluid and the number of passes have a significant impact on the efficiency of the cell breaking.

It is therefore recommended, according to the process in accordance with the invention, to work:
at a pressure between 300 and 400 MPa in at least one pass and preferably in a single pass, or
at a pressure between 150 and 300 MPa, preferably between 150 and 250 MPa, in at least two successive passes, and
at a feed temperature between 4 and 40° C.

With this process, the percentage of cell-wall breaking is more than 80%, 85% or 90%. Moreover, the microbial load is significantly decreased by this process. In particular, it is less than 10 total microorganisms per g of emulsion.

In the one-pass embodiment, the pressure may be between 300 and 325 MPa, between 325 and 350 MPa, between 350 and 375 MPa, or between 375 and 400 MPa.

In the embodiment with several successive passes, the pressure may be between 150 and 175 MPa, between 175 and 200 MPa, between 200 and 225 MPa, between 225 and 250 MPa, between 250 and 275 MPa, or between 275 and 300 MPa. In particular, the pressure may be between 200 and 300 MPa or between 200 and 250 MPa.

The feed temperature may be between 20 and 40° C. Alternatively, it may be between 4 and 10° C., between 10 and 20° C., between 20 and 30° C. or between 30 and 40° C.

Evaluation of the Capacity of the HPH Technology to Generate an Emulsion of Fine Particles

As will be shown hereinafter, the optical microscopy observations, with or without staining, show that the technology consisting in breaking by HPH makes it possible to generate an emulsion that is finer than the one produced by a more conventional cell-breaking method, in the case in point using ball milling.

The emulsion fraction resulting from the HPH cell breaking is characterized by a very small particle size, whereas that resulting from ball milling is characterized by a coarser particle size of from 1 to more than 40 μm. Indeed, with the process according to the present invention, a population having a particle size of less than 1 μm is significantly present, and may even be predominant in the emulsion.

In one embodiment, the emulsion fraction resulting from the HPH cell breaking obtained by means of the process according to the present invention is characterized by the particle size of an emulsion prepared with this flour. The emulsions are then analyzed by laser particle size analysis (Laser Mastersizer 2000^{E}—Malvern).

Thus, the emulsion obtained by means of the process according to the present invention is characterized by a population having a particle size of less than 1 μm representing at least 20%, 30%, 40% or 50% of the total population. The term “%” is intended to mean herein the area distribution percentage in a graph representing the % volume as a function of particle size.

In one preferred embodiment, the emulsion obtained by means of the process according to the present invention is characterized by a population having a particle size of less than 1 μm representing more than 50% of the total population. The term “%” is intended to mean herein the area distribution percentage in a graph representing the % volume as a function of particle size.

In one particularly preferred embodiment, the process comprises at least two successive passes at a pressure between 150 and 300 MPa, preferably between 200 and 300 MPa, in particular 250 MPa, and the emulsion obtained by means of the process according to the present invention is characterized by a population having a particle size of less than 1 μm representing more than 50% of the total population.

By virtue of its particle size characteristic, the emulsion obtained by high-pressure homogenization is therefore much more stable than the one resulting from ball milling.

Although by using very high pressure, in particular at least 300 MPa, it has been demonstrated that a single pass is sufficient for the process for breaking microalgal cells; it has also been demonstrated by the applicant company that a very-high-pressure homogenization will result in an emulsion that is all the more stabilized if it is produced in several successive passes. Thus, the process may comprise two, three, four or five successive passes. However, for industrial purposes, it is preferable to limit the number of passes. Thus, in one preferred embodiment, two successive passes will be used. The pressure used for each pass can vary or can be constant.

The process can be carried out on any available high-pressure homogenizers, for example those provided by Microfluidics, Stansted Fluid Power, AVP, Avestin or Niro Soavi.

The present invention also relates to a process for preparing a flour of microalgae as described above, which comprises production of a microalgal biomass, cell-wall breaking by means of a process according to the present invention, and drying of the biomass, in particular by spray drying. Prior to the cell-wall breaking, it is possible to wash the biomass and/or to concentrate it. The present invention also relates to the microalgal flour obtained by means of the process described above.

More generally, the present invention relates to a method for preparing flour of microalgae as described above, using the process for breaking the cell wall of microalgae according to the present invention. It therefore relates to the use of the process for breaking the cell wall of microalgae according to the present invention, for preparing a flour of microalgae as described above.

This microalgal flour is of use in the food sector. Thus, the present invention also relates to the use of the flour according to the present invention or obtained by means of the process according to the present invention in the food sectors. In particular, it relates to a method for preparing a food composition comprising the addition of such a microalgal flour to ingredients of the food composition or to the food composition. Such uses are, for example, described in patent applications WO 2010/045368, WO 2010/120923 or US 2010/0297296.

The term “industrial scale” is preferably intended to mean a process in which:
the volume of the milling or breaking chamber is greater than or equal to 100 liters, preferably greater than or equal to 500 liters; and/or
the flow rate is greater than 1 m³/h; and/or
a batch is from 1 to 200 m³.

In addition, in one preferred industrial scale mode, the biomass has 15% to 50% by weight of solids, particularly by dry weight of cells.

The invention will be understood more clearly from the examples which follow, which are intended to be illustrative and nonlimiting.

### EXAMPLES

### Example 1

### Preparation of a Biomass of Chlorella Protothecoides Microalgae and Presentation of the Tools Used

The fermentation protocol is adapted from the one described entirely generally in patent application WO 2010/120923.

The production fermenter is inoculated with a pre-culture of Chlorella protothecoides. The volume after inoculation reaches 9000 l.

The carbon source used is a 55% w/w glucose syrup sterilized by application of a time/temperature scheme.

The fermentation is a fed-batch fermentation during which the glucose flow rate is adjusted so as to maintain a residual glucose concentration of from 3 to 10 g/l.

The production fermenter time is from 4 to 5 days.

At the end of fermentation, the cell concentration reaches 185 g/l.

During the glucose feed phase, the nitrogen content in the culture medium is limited so as to allow the accumulation of lipids in an amount of 50% (by weight of biomass).

The fermentation temperature is maintained at 28° C.

The fermentation pH before inoculation is adjusted to 6.8 and is then regulated on this same value during the fermentation.

The dissolved oxygen is maintained at a minimum of 30% by controlling the aeration, the counter pressure and the stirring of the fermenter.

The fermentation must is heat-treated over an HTST zone with a scheme of 1 min at 75° C. and cooled to 6° C.

The biomass is then washed with decarbonated drinking water with a dilution ratio of 6 to 1 (water/must) and concentrated to 250 g/l (25% DCW “Dry Cell Weight”) by centrifugation using an Alfa Laval Feux 510.

Measurement of the Degree of Cell Breaking

The degree of milling is measured by microscopic counting of the residual cells after milling, relative to the initial reference sample.

The samples are diluted to 1/800.

The analysis is carried out by counting on a Malassez cell according to the standard method of use under an optical microscope at a magnification of 10×40.

The degree of cell breaking is determined by calculating the percentage of residual cells relative to the initial reference sample.

### Example 2

### Measurement of the Impact of the Pressure Values, of the Number of Passes and of the Temperature on the Efficiency of the Breaking of Chlorella Protothecoides by HPH

The impact of the value of the pressure applied (100 MPa and 150 MPa), and the number of passes, on the efficiency of milling, expressed as % degree of breaking, of the biomass prepared according to example 1, is tested.

A Rannie LAB 10.51VH homogenizer (SPX) high-pressure homogenization system with an SEO valve is used at a dynamic pressure ranging up to 150 MPa. The biomass is introduced at a flow rate of approximately 60 l/h.

FIG. 1 clearly shows that the higher the pressure and the number of passes, the better the breaking efficiency will be.

A second series of tests is carried out in a single pass, but at various pressures in order to evaluate the efficiency of the cell breaking according to the pressure applied.

To do this, a Stansted Fluid Power Ltd 11300 (15 kW) continuous ultrahigh pressure system is used at a dynamic pressure ranging up to 380 MPa.

The product is continuously fed at a flow rate of approximately 100 l/h in a single pass.

As shown in FIG. 2, the breaking of the cells is visible starting from 100 MPa and begins to be significant starting from 150 MPa.

The degree of cell breaking passes 70% at 300 MPa and reaches a degree above 80% at the maximum pressure attainable using the technology.

A third series of tests is carried out by making two successive passes, but limiting the pressure to 250 MPa.

As shown in FIG. 3, in this configuration, by accumulating two successive passes at pressures above 200 MPa, degrees of cell breaking of about 90% are obtained.

In this configuration, it is possible to limit the pressure applied to the homogenization valve to a value of less than 250 MPa.

This scheme makes it possible to limit homogenization-valve wear phenomena.

The impact of the input temperature on the cell-breaking efficiency is evaluated in another series of tests.

An increase in the product feed temperature acts favorably on the cell-breaking efficiency (gain of more than 5%)—cf. FIG. 4.

However, the exothermicity of the high-pressure homogenization process generates a very significant increase in temperature which can constitute a constraint according to the sensitivity of the product at output; the input temperature is therefore to be limited according to the maximum tolerable threshold at output.

In conclusion, it is recommended, according to one preferred mode of the process in accordance with the invention, to work:
at a pressure between 350 and 400 MPa in a single pass, or
at a pressure between 200 and 250 MPa in two successive passes,
at a feed temperature between 4 and 40° C.

### Example 3

### Quality of the Emulsion Generated by the HPH Breaking

The composition of the biomass resulting from the fermentation according to example 1 is characterized by a predominant lipid fraction (approx. 50%/dry).

After cell breaking, an emulsion (aqueous phase with cell debris/oil) is thus generated.

The stability of this emulsion is conditioned by the fineness of the lipid globules.

The objective of the homogenization is to minimize the diameter of the lipid globules and at the same time to make them as uniform as possible; this then results in an improvement in the stability and an increase in the viscosity of the medium.

The high-pressure homogenization technology is thus evaluated, compared with the ball-milling technology, with respect to its potential to homogenize and thus stabilize the emulsion generated beyond the simple objective of cell breaking.

The biomass is then milled with a Netzsch Labstar ball mill using zirconium silicate balls 0.5 mm in diameter (peripheral speed: 12 m/s, 90% filling rate, flow rate: 6 kg/h).

An evaluation under an optical microscope is then carried out in order to compare the emulsion according to the process used.

FIGS. 5 and 6 make it possible to visualize the difference in size of the emulsion generated by the two technologies (HPH carried out in one pass, at 350 MPa, and ball milling).

FIG. 6 demonstrates the fact that the ball milling leads to the production of a much coarser emulsion with a globule size that is larger and more heterogeneous than that obtained after HPH processing (FIG. 5); the emulsion generated is visually much finer.

In order to more precisely characterize the emulsification potential of the HPH technology compared with what is obtained by ball milling, the emulsions generated are analyzed on a laser particle size analyzer (Laser Mastersizer 2000^{E}—Malvern) according to the constructor's specifications.

FIG. 7 makes it possible to characterize the efficiency of the methods used in the generation of a fine and potentially stable emulsion.

After cell breaking, in the two cases, a bimodal population is observed compared with the initial biomass which has, for its part, a monomodal population, with a whole-cell diameter distributed between 1 and 10 μm.

However, in the case of the ball milling, in accordance with the microscopic observations, the emulsion is coarse, distributed between 1 and 100 μm.

That is to say that the 1st population, between 1 and 30 μm, consists of residual whole cells and also of lipid globules with a size <30 μm.

The 2nd population, for its part, consists of an emulsion consisting of lipid globules with a diameter greater than 40 μm.

Overall, the emulsion resulting from the ball milling is therefore coarse and very heterogeneous.

In the case of the cell breaking by high-pressure homogenization, it is the 2nd population, distributed between 1 and 10 μm, which consists exclusively of residual whole cells.

The emulsion fraction resulting from the cell breaking is characterized as 1st population, by a very small particle size, less than 1 μm.

By virtue of its particle size characteristic, the emulsion obtained by high-pressure homogenization is therefore much more stable than the one resulting from ball milling.

FIG. 8 subsequently demonstrates the impact of the homogenization pressure on the particle size distribution of the emulsion.

It is in fact noted that the higher the pressure applied, the lower the amount of residual whole cells which explains the reduction in the magnitude of the corresponding 1 μm and 10 μm population.

The magnitude of the fraction corresponding to the emulsified lipid globules then increases with the increase in the pressure, added to which is a reduction in the average particle size distribution.

Additional tests, illustrated by FIG. 9, aimed to evaluate the system implementing one or two high-pressure (250 MPa) passes.

It is thus noted that the influence of the pressure is all the greater when the homogenization process consists of two successive passes at very high pressure than when it consists of a single pass (thus generating a cumulative pressure that is even greater than the maximum attainable pressure with a single pass).

By virtue of these very-high-pressure homogenization configurations, the lipid emulsion resulting from the cell breaking is greatly stabilized, thereby facilitating the rest of the operations for processing this emulsion and also its use in applications.

### Example 4

### Reduction of the Microbial Load

The very-high-pressure processing is evaluated with respect to its potential to reduce the contamination of the biomass before homogenization.

The shear generated by the pressure on the valve of the homogenizer, combined with the increase in temperature, significantly reduces the microbial load of the biomass and thus generates a sterilizing force.

Thus, the decontamination potential is evaluated with a different pressure scheme on the Stansted Fluid Power Ltd 11300 (15 kW) continuous ultrahigh pressure system.

The following table presents the microbial load results obtained under these conditions.

| | Initial | HPH | HPH |
|---|---|---|---|
| | point | 250 MPa | 350 MPa |
| Output T° C. | 8 | 72 | 91 |
| total microorgan- | 42000 | <10 | <10 |
| isms at 30° C. | | | |

The very-high-pressure homogenization allows a significant reduction in the microbial load by allowing a virtually total microbial reduction close to sterilization.

### DESCRIPTION OF THE FIGURES

FIG. 1: Evaluation of the degree of breaking according to the pressure (100 MPa/150 MPa) and the number of passes.

FIG. 2: Evaluation of the degree of breaking according to the pressure in a single pass.

FIG. 3: Evaluation of the degree of breaking according to the pressure in two successive passes.

FIG. 4: Evaluation of the degree of breaking as a function of the temperature of the product at feed.

FIG. 5: Microscopic observation of the emulsion resulting from the ball milling (Magnification×760, scale: bar=20 μm).

1—White light

2—Staining with Nile blue and fluorescent light

FIG. 6: Microscopic observation of the emulsion resulting from the HPH breaking at 350 MPa (Magnification×760, scale: bar=20 μm).

1—White light

2—Staining with Nile blue and fluorescent light

FIG. 7: Particle size analysis of the emulsion produced by ball milling or by HPH relative to the initial biomass.

FIG. 8: Particle size analysis of the emulsion produced by HPH according to the pressure applied.

FIG. 9: Particle size analysis of the emulsion produced by HPH in 1 or 2 passes at 250 MPa.

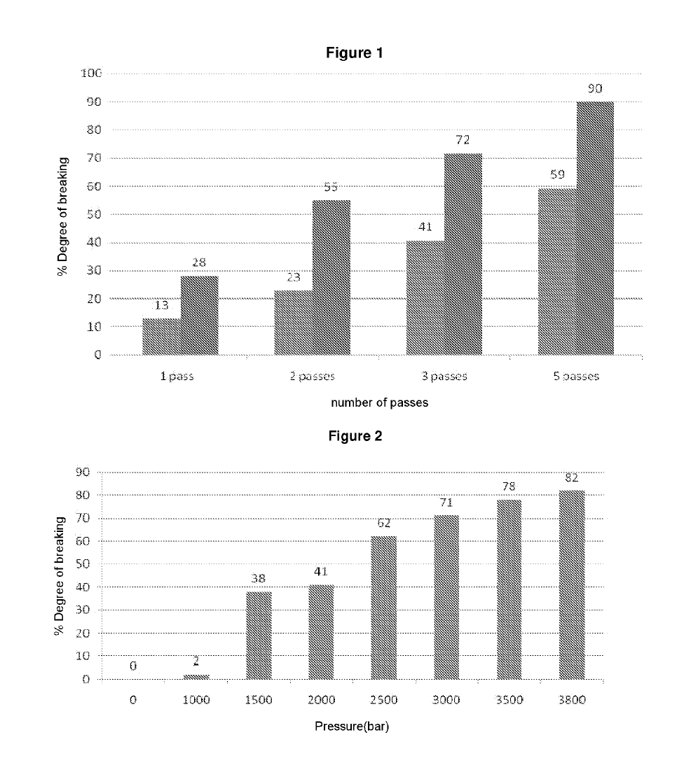

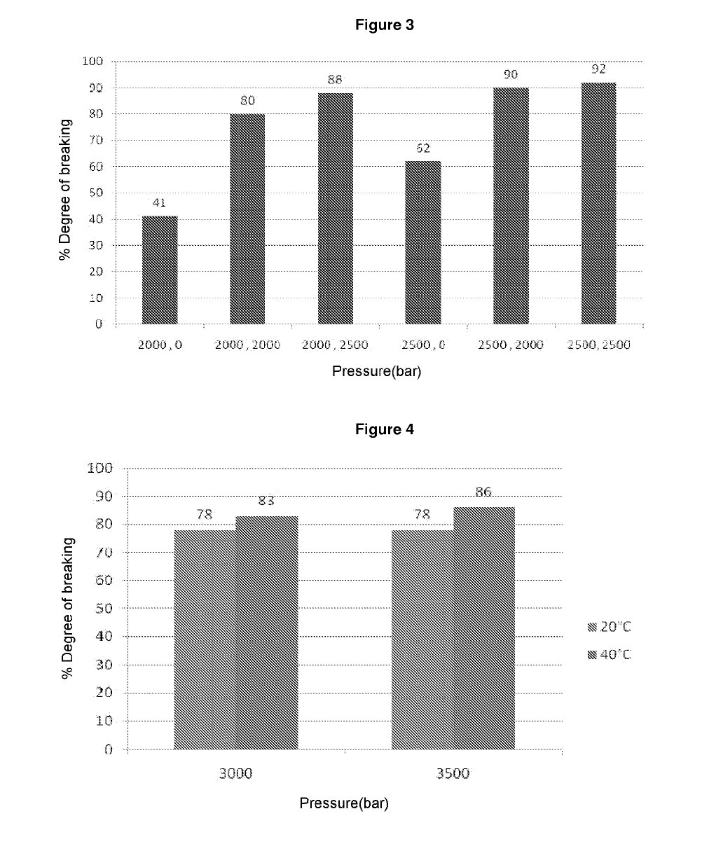

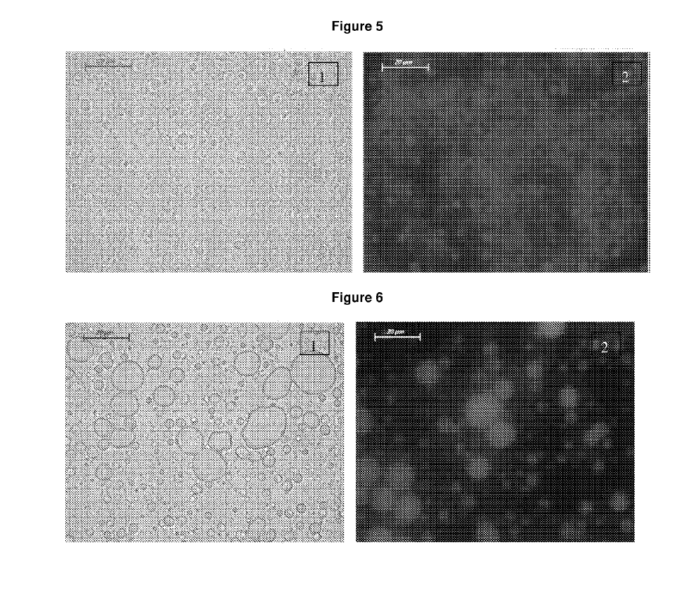

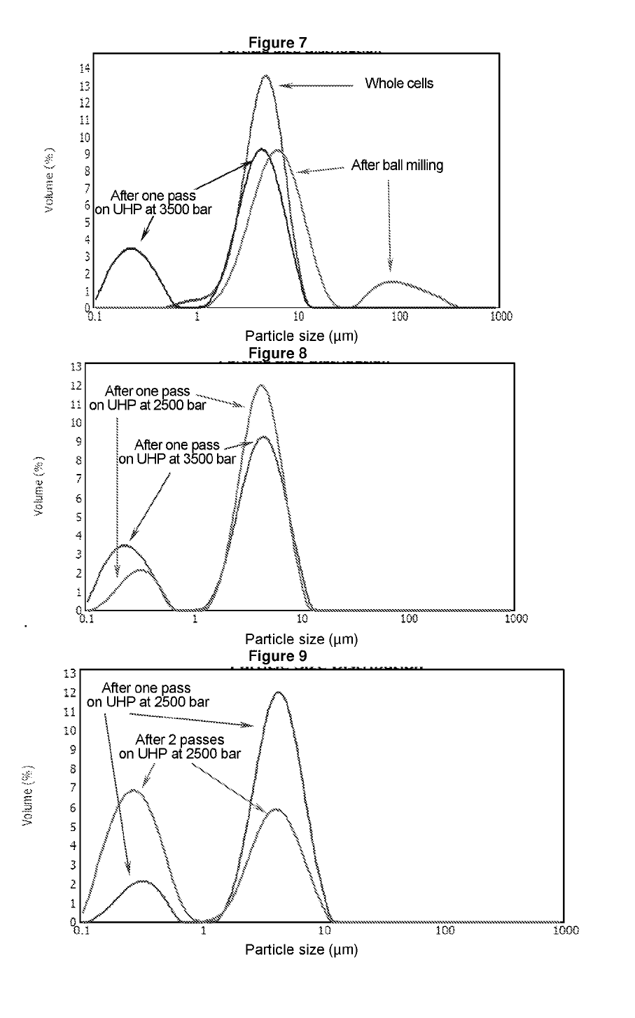

1-12. (canceled)

13. A process for breaking the cell wall, on an industrial scale, of cells of microalgae of the Chlorella genus, the cell-wall breaking being carried out by high-pressure homogenization, wherein the high-pressure homogenization is carried out:
in at least one pass at a pressure between 300 and 400 MPa, or
in at least two successive passes at a pressure between 150 and 300 MPa,
at a feed temperature between 4 and 40° C., and
with a biomass of microalgal cells comprising between 15%
and 50% by weight of solids;
in such a way as to guarantee a degree of cell-wall breaking of more than 80%, a particle size of the emulsion produced of less than 1 μm and a decrease in microbial load by a factor of 1000.

14. The process as claimed in claim 13, wherein the microalgae of the Chlorella genus are chosen from the group consisting of Chlorella vulgaris, Chlorella sorokiniana and Chlorella protothecoides, and are more particularly Chlorella protothecoides.

15. The process as claimed in claim 13, which guarantees a microbial load of less than 10 total microorganisms per g of emulsion.

16. The process as claimed in claim 13, wherein the high-pressure homogenization is carried out at a pressure between 300 and 400 MPa in a single pass.

17. The process as claimed in claim 13, wherein the high-pressure homogenization is carried out at a pressure between 150 and 250 MPa in two, three, four or five successive passes.

18. The process as claimed in claim 17, wherein the high-pressure homogenization is carried out at a pressure between 150 and 250 MPa in two successive passes.

19. The process as claimed in claim 13, wherein the pressures of two successive passes may be identical or different.

20. The process as claimed in claim 13, wherein the feed temperature is between 20 and 40° C.

21. The process as claimed in claim 13, wherein the microalgal biomass comprises at least 10% by dry weight of lipids.

22. A process for preparing a microalgal flour, which comprises production of a microalgal biomass, cell-wall breaking by means of a process as claimed in claim 13, and drying of the biomass, in particular by spray drying.

23. A microalgal flour obtained by means of the process as claimed in claim 22, said process comprising at least two successive passes at a pressure between 150 and 300 MPa.

24. A process for preparing a microalgal flour, which comprises breaking the cell wall of microalgae according to the process as claimed in claim 13.

25. The process as claimed in claim 13, wherein the microalgal biomass comprises at least 20% by dry weight of lipids.

26. The process as claimed in claim 13, wherein the microalgal biomass comprises at least 30% by dry weight of lipids.

27. The process as claimed in claim 13, wherein the microalgal biomass comprises at least 40% by dry weight of lipids.

28. The process as claimed in claim 13, wherein the microalgal biomass comprises at least 50% by dry weight of lipids.

29. The process as claimed in claim 13, wherein the microalgal biomass comprises at least 60% by dry weight of lipids.
